(19) United States
(12) Plant Patent Application Publication
(10) Pub. No.: US 2012/0246780 P1
(43) Pub. Date: Sep. 27, 2012

(54) Verbena plant named 'KLEVP10410'

(50) ;

(75) Inventor: Ruijun Li, North Parramata (AU)

(73) Assignee: KLEMM+SOHN GMBH & CO. KG, Stuttgart (DE)

(21) Appl. No.: 13/065,432

(22) Filed: Mar. 22, 2011

(51) Int. Cl. A01H 5/00

(52) U.S. Cl. PLT/308

(57) ABSTRACT

A new variety of Verbena named ‘KLEVP10410’, particularly characterized by brilliant white colored flowers, compact growth habit, good center branching, and good mildew resistance, is disclosed.

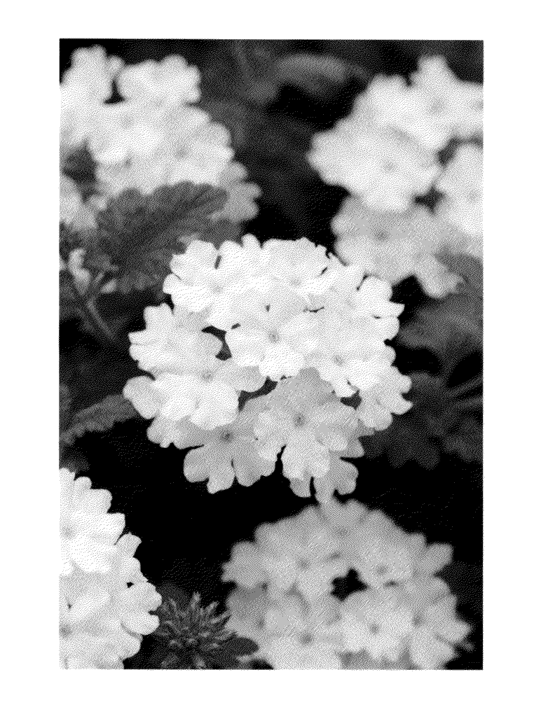

### GENUS AND SPECIES

Verbena×hybrida

### VARIETY DENOMINATION

‘KLEVP10410’

### BACKGROUND OF THE NEW PLANT

The present invention comprises a new and distinct variety of Verbena, botanically known as Verbena×hybrida, and hereinafter referred to by the variety name ‘KLEVP10410’. The new variety is the result of a cross conducted in July 2007 in Cobbity, New South Wales, Australia between the proprietary female Verbena parent, ‘06.00.21’, and the proprietary male Verbena parent, ‘06.00.24’. A single plant selection was subsequently chosen for further evaluation and asexual propagation.

The new variety was first propagated in January 2008 in Cobbity, New South Wales, Australia and has been asexually reproduced repeatedly by vegetative cuttings over three to four generations. ‘KLEVP10410’ has been found to retain its distinctive characteristics through successive asexual propagations.

No Plant Breeder's Rights have been applied for this variety. ‘KLEVP10410’ has not been made publicly available more than one year prior to filing of this application.

### SUMMARY OF THE INVENTION

The following are the most outstanding and distinguishing characteristics of this new variety when grown under normal horticultural practices in a greenhouse in Stuttgart, Germany.

1. Brilliant white colored flowers;

2. Compact growth habit; and

3. Good center branching.

### DESCRIPTION OF THE PHOTOGRAPH

This new Verbena plant is illustrated by the accompanying photograph which shows inflorescences and foliage of the plant. The colors shown are as true as can be reasonably obtained by conventional photographic procedures. The photograph was taken in Spring 2010 of a plant about 3 months old grown from a rooted cutting in a glass greenhouse in Stuttgart, Germany under normal horticultural practices.

### DESCRIPTION OF THE NEW VARIETY

The following detailed descriptions set forth the distinctive characteristics of ‘KLEVP10410’. The data which define these characteristics were collected from asexual reproductions carried out in Stuttgart, Germany. The plant history was taken in Spring 2010 on 3-month old plants grown in 12 centimeter pots in a glass greenhouse with a minimum pinch date of 10 weeks. The color readings were determined under natural light. Color references are to the RHS Colour Chart of The Royal Horticultural Society of London (R.H.S), Fifth Edition (2007).

### DESCRIPTION OF THE NEW PLANT

Classification:
   Family.—Verbenaceae.
   Genus and species.—Verbena×hybrida.
   Common name.—Verbena.
Parentage:
   Female parent.—The proprietary Verbena plant, ‘06.00.21.’
   Male parent.—The proprietary Verbena plant, ‘06.00.24.’
Plant:
   Form.—Semi-trailing.
   Growth and branching habit.—Freely branching.
   Height (from top of soil).—Approximately 12.0 cm.
   Width (horizontal plant diameter).—Approximately 30.0 cm.
   Time to produce a finished flowering plant.—Approximately 11 weeks.
   Time to initiate and develop roots.—3 weeks.
   Root description.—White with normal structure.
Stems:
   Length.—Approximately 15.0 cm.
   Diameter per branch (measured from the midpoint).—0.2 cm.
   Internode length.—1.0 cm.
   Color.—RHS 131D.
   Anthocyanin.—Absent.
   Texture and appearance.—Little pubescence.
Leaves:
   Arrangement.—Opposite.
   Color.—Immature and mature leaves (both surfaces): RHS 141A.
   Length.—2.5 cm.
   Width.13 1.5 cm.
   Shape.—Elliptic.
   Apex.—Cuspidate.
   Base.—Closed.
   Margin.—Crenate.
   Texture.—Leathery.
   Venation.—Palmate.
   Venation color.—RHS 141A.
   Petioles.—Length: 0.5 cm Diameter: 0.1 cm Color: RHS 141A Texture: Rough.
Flower buds:
   Shape.—Tall cylinder.
   Length.—1.0 cm.
   Diameter.—0.2 cm.
   Color (at tight bud).—RHS 131D.
Inflorescence:
   Blooming habit (flowering season).—From early Spring to late Fall in Middle Europe.
   Lastingness of individual blooms on the plant.—1 to 2 weeks.
   Fragrance.—Absent.
   Inflorescence type.—Umbel.
   Length of inflorescence.—5.0 cm.
   Diameter of inflorescence.—5.0 cm.
   Number of florets per inflorescence.—More than 10.
   Peduncle.—Length: 3.0 cm Diameter: 0.1 cm Color: RHS 141A Texture: Rough.
Florets:
   Form.—Tall cylinder, opening at the end.
   Length.—1.5 cm.
   Diameter.—0.2 cm at the bottom, 1.5 cm at the top.
   Color.—Immature and mature flowers (both surfaces): RHS 155C.
   Corolla tube length.—1.5 cm.
   Corolla tube color.—RHS N155B.
   Petals.—Length of lobe: 0.5 cm Diameter of lobe: 0.5 cm Lope shape: Reniform Apex: Obtuse Base: Closed Margin: Entire.
   Pedicels.—Length: 0.5 cm Diameter: 0.5 cm Color: RHS 155C Texture and appearance: Smooth and satiny.
Calyx:
   Arrangement.—Round.
Sepals:
   Shape.—Funnel.
   Length.—1.5 cm.
   Diameter.—0.2 cm.
   Color.—RHS 157B.
   Apex.—Wide open.
   Base.—Open.
   Margin.—Satiny.
   Texture.—Smooth.
Reproductive organs:
   Stamens.—Quantity: 5 Color: RHS N155D Filament length: 0.5 cm Filament diameter: Less than 0.1 cm.
   Anther.—Shape: Fused Color: RHS 155D Length: 0.2 cm Diameter: Less than 0.1 cm.
   Pollen.—Amount: Sparse.
   Pistils.—Number: 1 Length: Less than 0.1 cm Diameter: Less than 0.1 cm Stigma: Color: RHS 155C Shape: Lanceolate Length: Less than 0.5 cm Diameter: Less than 0.1 cm Style: Color: RHS 155C Length: Less than 0.3 cm Diameter: Less than 0.1 cm.
Fruit and seed set: None observed.
Disease, insect resistance, and physiological stress: Resistance against powdery mildew.
Comparision with parental and commercial varieties: ‘KLEVP10410’ differs from the female parent, the proprietary Verbena ‘06.00.21’, in that ‘KLEVP10410’ has a more compact growth habit, while ‘06.00.21’ has a strong growth habit. ‘KLEVP10410’ differs from the male parent, the proprietary Verbena ‘06.00.24’, in that ‘KLEVP10410’ has a compact growth habit, while ‘06.00.24’ has a very compact growth habit. ‘KLEVP10410’ differs from the commercial comparison variety, ‘Lascar Compact White’ (unpatented), in that ‘KLEVP10410’ has a semi-trailing growth habit, while ‘Lascar Compact White’ has a more upright growth habit.

I claim:

1. A new and distinct variety of Verbena plant named ‘KLEVP10410’ as shown and described herein.
